(19) United States
(12) Patent Application Publication
(10) Pub. No.: US 2011/0301644 A1
(43) Pub. Date: Dec. 8, 2011

(54) SPINAL STABILIZATION SYSTEM

(75) Inventor: Karl Pierre Belliard, La Membrolle (FR)

(73) Assignee: Zimmer Spine, Bordeaux (FR)

(21) Appl. No.: 12/796,004

(22) Filed: Jun. 8, 2010

(51) Int. Cl. A61B 17/70; A61B 17/88

(52) U.S. Cl. 606/263; 606/264; 606/279

(57) ABSTRACT

A spinal stabilization system for a spinal column including a flexible support construct extending between a first vertebral connector configured to be secured to a first vertebra and a second vertebral connector configured to be secured to a second vertebra. At least one of the vertebral connectors includes an elongate band configured to pass around a bony portion of a vertebra, such as a transverse process, to secure the vertebral connector to the vertebra without invasively removing material from the vertebra and/or penetrating into the vertebra.

### TECHNICAL FIELD

The disclosure is directed to a vertebral stabilization system. More particularly, the disclosure is directed to a spinal stabilization system which can be secured to one or more vertebrae of a spinal column without pedicle screws.

### BACKGROUND

The spinal column of a patient includes a plurality of vertebrae linked to one another by facet joints and an intervertebral disc located between adjacent vertebrae. The facet joints and intervertebral disc allow one vertebra to move relative to an adjacent vertebra, providing the spinal column a range of motion. Diseased, degenerated, damaged, or otherwise impaired facet joints and/or intervertebral discs may cause the patient to experience pain or discomfort and/or loss of motion, thus prompting surgery to alleviate the pain and/or restore motion of the spinal column.

One possible method of treating these conditions is to immobilize a portion of the spine to allow treatment. Traditionally, immobilization has been accomplished by rigid stabilization. For example, in a conventional spinal fusion procedure, a surgeon restores the alignment of the spine or the disc space between vertebrae by installing a rigid fixation rod between pedicle screws secured to adjacent vertebrae. Bone graft is placed between the vertebrae, and the fixation rod cooperates with the screws to immobilize the two vertebrae relative to each other so that the bone graft may fuse with the vertebrae.

Dynamic stabilization has also been used in spinal treatment procedures. Dynamic stabilization does not result in complete immobilization, but instead permits a degree of mobility of the spine while also providing sufficient support and stabilization to effect treatment. One example of a dynamic stabilization system is the Dynesys® system available from Zimmer Spine, Inc. of Minneapolis, Minn. Such dynamic stabilization systems typically include a flexible member positioned between pedicle screws installed in adjacent vertebrae of the spine. A flexible cord can be threaded through the bore in the flexible member and secured to the pedicle screws while cooperating with the flexible member to permit mobility of the spine.

In some instances, however, it may be desirable or necessary to secure a dynamic stabilization construct to one or more vertebrae of a spinal segment without the use of a pedicle screw. Accordingly, it may be desirable to provide alternative spinal stabilization systems which may be secured to one or more vertebrae of a spinal segment of a spinal column without the use of a pedicle screw.

### SUMMARY

The disclosure is directed to several alternative designs, materials and methods of manufacturing medical device structures and assemblies and uses thereof.

Accordingly, one illustrative embodiment is a spinal stabilization system for a spinal column including a first vertebral connector configured to be secured to a first vertebra and a second vertebral connector configured to be secured to a second vertebra. The spinal stabilization system further includes a support construct positionable between the first vertebral connector and the second vertebral connector for providing stabilization between the first and second vertebrae. The support construct includes a flexible cord extending from an insert and a spacer including a lumen through which the cord extends through. The first vertebral connector includes a connector body having a channel for receiving the insert therein and an elongate band having a first end, a second end and a portion, such as a loop portion, configured to pass around a bony portion of the first vertebra to secure the first vertebral connector to the first vertebra.

Another illustrative embodiment is a spinal stabilization system for a spinal column. The system includes first and second vertebral connectors, first and second inserts, a spacer and a flexible member extending through the spacer. The first vertebral connector is configured to be secured to a first vertebra. The first vertebral connector includes a connector body having a channel extending through the connector body from a first end of the connector body to a second end of the connector body and an elongate band having a first end, a second end and a portion, such as a loop portion, configured to pass around a bony portion of the first vertebra to secure the first vertebral connector to the first vertebra. The second vertebral connector is configured to be secured to a second vertebra. The second vertebral connector includes a connector body having a channel extending through the connector body from a first end of the connector body to a second end of the connector body. The first insert includes a first flange, a second flange and a medial portion extending between the first flange and the second flange, with the medial portion of the first insert configured to be positioned in the channel of the connector body of the first vertebral connector while the first flange is positioned on a first side of the connector body and the second flange is positioned on a second side of the connector body opposite the first side. The second insert includes a first flange, a second flange and a medial portion extending between the first flange and the second flange, with the medial portion of the second insert configured to be positioned in the channel of the connector body of the second vertebral connector while the first flange is positioned on a first side of the connector body and the second flange is positioned on a second side of the connector body opposite the first side. The spacer is positionable between the first insert and the second insert with the flexible member extending through the spacer from the first insert to the second insert.

Another illustrative embodiment is a spinal stabilization system including a first vertebral connector configured to be secured to a first vertebra and a second vertebral connector configured to be secured to a second vertebra. The first vertebral connector includes a connector body having a channel extending through the connector body from a first end of the connector body to a second end of the connector body, and a flexible component configured to pass around a bony portion of the first vertebra to secure the first vertebral connector to the first vertebra. The second connector includes a connector body having a channel extending through the connector body from a first end of the connector body to a second end of the connector body. The system also includes a first insert including at least a portion positionable in the channel of the connector body of the first vertebral connector, and a second insert including at least a portion positionable in the channel of the connector body of the second vertebral connector. The system also includes a spacer positionable between the first insert and the second insert, and a flexible member extending through the spacer from the first insert to the second insert.

Yet another illustrative embodiment is a method of stabilizing a spinal segment of a spinal column. The method includes securing a first vertebral connector to a first vertebra of the spinal column by passing a flexible band of the first vertebral connector around a bony portion of the first vertebra and securing the flexible band to a connector body of the first vertebral connector. A second vertebral connector is secured to a second vertebra of the spinal column. A stabilization construct, which is configured to allow a range of flexion and extension of the spinal segment, is coupled between the first vertebral connector and the second vertebral connector.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a perspective view of an exemplary spinal stabilization system;

FIG. 2 is an exploded view of the spinal stabilization system of FIG. 1;

FIGS. 3 and 3A are top and cross-sectional views, respectively, of the connector body of the vertebral connector of FIGS. 1 and 2;

FIGS. 4A and 4B illustrate two alternate ways of routing a band through the connector body of the vertebral connector during installation of the vertebral connector on a vertebra;

FIG. 5 illustrates the vertebral connector secured to a vertebra;

FIG. 6 is a perspective view of the spinal stabilization system of FIGS. 1 and 2 installed on a spinal segment;

FIG. 7 is a perspective view of an exemplary multi-level spinal stabilization system;

FIG. 8 is a perspective view of another exemplary spinal stabilization system;

FIG. 9 is a perspective view of an insert used with the spinal stabilization system of FIG. 8;

FIGS. 10A and 10B illustrate two possible ways to apply a clamping force on the insert of FIG. 9; and

FIGS. 11A and 11B illustrate the possible effects of applying a clamping force on the insert of FIG. 9 to clamp a flexible member therein.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

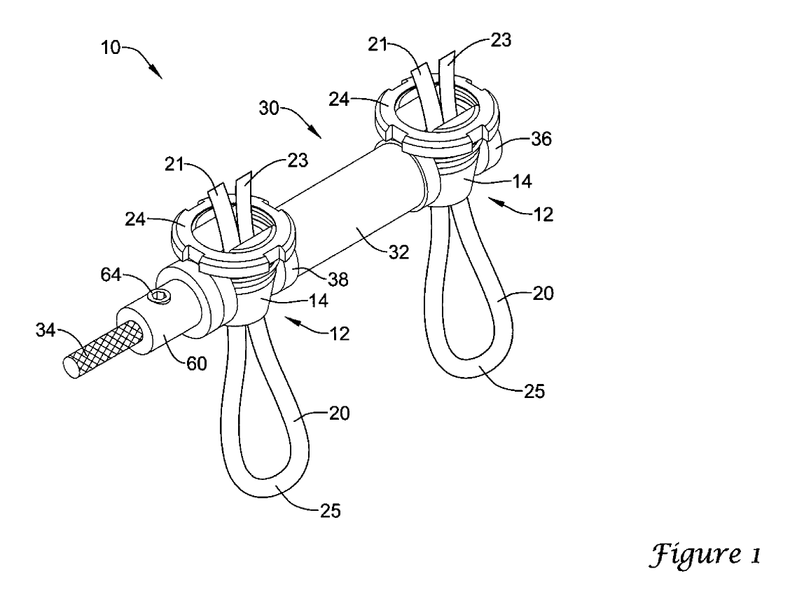

Referring now to FIG. 1, there is shown a spinal stabilization system 10 for stabilizing a portion of a spinal column, such as one or more spinal segments of a spinal column. As used herein, a spinal segment is intended to refer to two or more vertebrae, the intervertebral disc(s) between the vertebrae and other anatomical elements between the vertebrae. For example, a spinal segment may include first and second adjacent vertebrae and the intervertebral disc located between the first and second vertebrae. The spinal stabilization system 10, in some instances, may provide dynamic stabilization to a spinal segment, preserving and/or allowing for a range of motion of the spinal segment. For example, in some instances the spinal stabilization system 10 may allow for a range of motion of greater than 0 degrees to about 15 degrees or more, greater than 0 degrees to about 10 degrees or more, or greater than 0 degrees to about 5 degrees or more between adjacent vertebrae. In some instances, the spinal stabilization system 10 may allow for 2 degrees or more, 5 degrees or more, 10 degrees or more, or 12 degrees or more of motion between adjacent vertebrae.

In some embodiments, the spinal stabilization system 10 may be used to treat discogenic low back pain, degenerative spinal stenosis, disc herniations, facet syndrome, posterior element instability, adjacent level syndrome associated with spinal fusion, and/or other maladies associated with the spinal column.

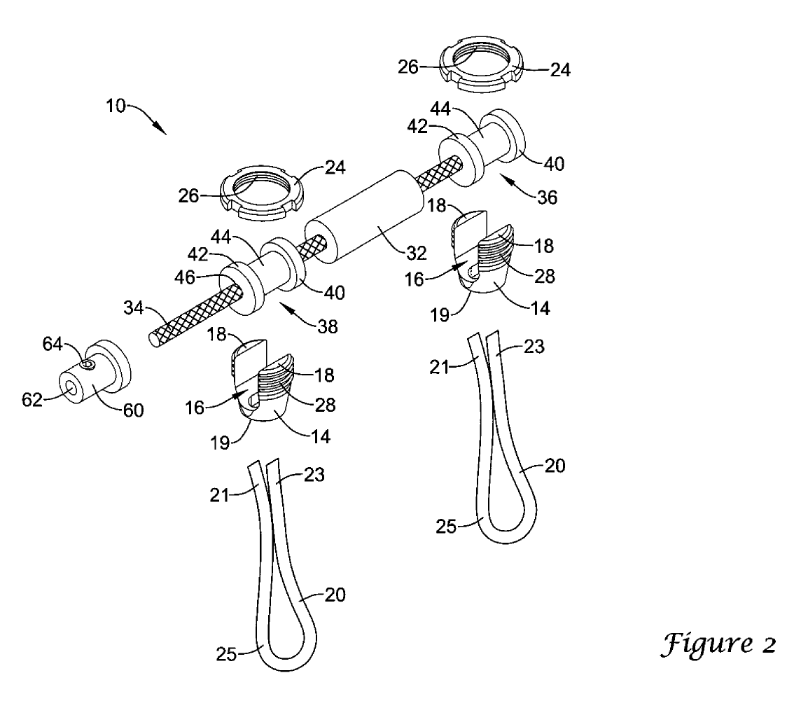

The spinal stabilization system 10 may include one or more or a plurality of vertebral connectors for securing the spinal stabilization system 10 to one or more vertebrae of the spinal segment to be stabilized. One or more of the vertebral connectors, as shown in FIGS. 1 and 2, may be vertebral connectors (ref. number 12) which include a band 20, or other flexible member, for wrapping around a bony structure of a vertebra (e.g. transverse process, costal process, spinous process, vertebral arch, etc.) to secure the vertebral connector 12 to a vertebra of the spinal column. Thus, the vertebral connector 12 may be secured to a vertebra without invasively removing material from the vertebra and/or penetrating into the vertebra.

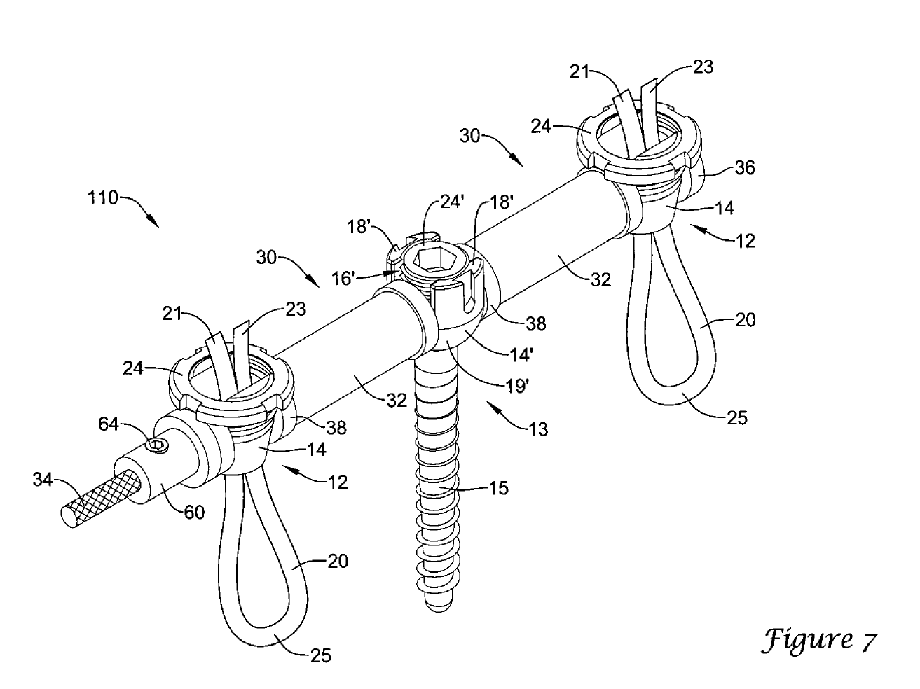

However, in some embodiments one or more of the vertebral connectors may be pedicle screws 13 (one of which is shown in FIG. 7), vertebral hooks (e.g., laminar hooks) or other types of fastening members for attachment to a bony structure such as a vertebra of the spinal column. For instance, the pedicle screw 13 may be screwed into the pedicle of a vertebra to secure the stabilization system 10 to the spinal column while one or more of the other vertebral connectors of the stabilization system 10 may be a vertebral connector 12 including a band 20 for securing the vertebral connector 12 to the vertebra. It is noted however that the pedicle screw 13 may be replaced with another vertebral connector 12 having a band 20 for securing the vertebral connector 12 to a vertebra, if desired.

In some instances, the vertebral connector 12 may be similar to those disclosed in U.S. Pat. App. Pub. 2009/0105715, incorporated by reference herein. For instance, the vertebral connector 12 may include a connector body 14 which may include first and second legs 18 extending from a base portion 19 defining a channel 16 therebetween extending through the connector body 14 from a first side of the connector body 14 to a second side of the connector body 14 opposite the first side.

The vertebral connector 12 may also include a flexible member, such as a band 20, configured to be passed around a bony portion of a vertebra to secure the vertebral connector 12 to a vertebra of the spinal column. For example, the band 20 may include a first end portion 21 proximate a first end, a second end portion 23 proximate a second end, and a loop portion 25 between the first end portion 21 and the second end portion 23. The loop portion 25 may be wrapped around a bony portion of a vertebra and may be conformable to the contour of the periphery of the bony portion of the vertebra. The band 20 may be flat in some instances, having a width substantially greater than a thickness of the band 20. However, in other instances the band 20 may have a circular, semi-circular, oval, or other cross-sectional shape, if desired.

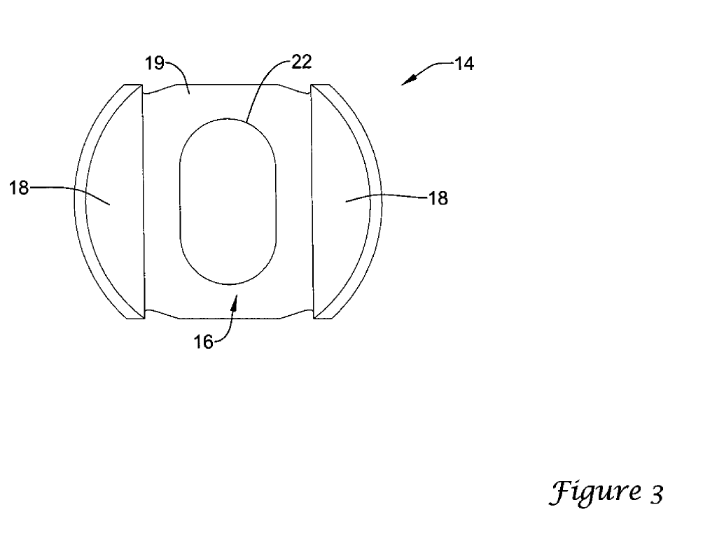

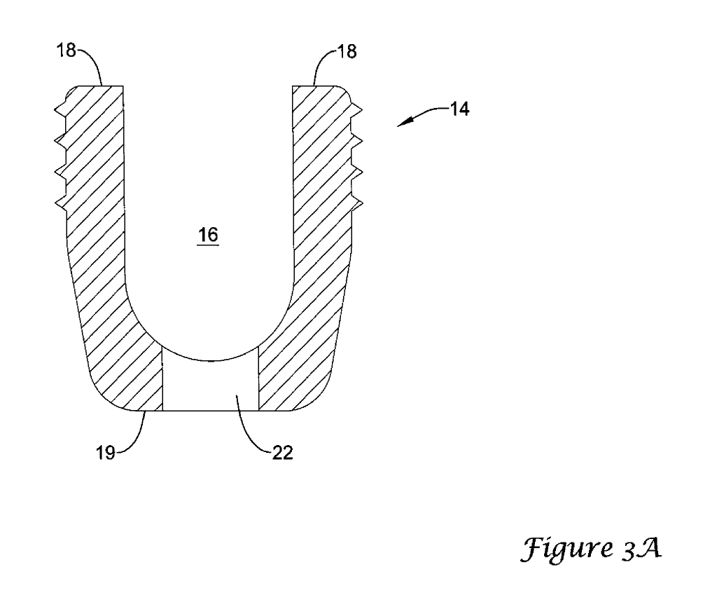

The connector body 14, as shown in FIGS. 3 and 3A, may include an opening 22 extending through the base portion 19 into the channel 16. Two portions of the band 20 may extend through the opening 22, such that the loop portion 25 extends from the opening 22 in a first direction, and first and second end portions 21, 23 extending to the first and second ends extend from the opening 22 in a second direction opposite the first direction.

The vertebral connector 12 may also include a securing element, such as a threaded fastener configured to rotatably engage the connector body 14 to secure a portion of a support construct 30 in the channel 16 of the connector body 14. As shown in FIGS. 1 and 2, the securing element may be a clamping ring 24 having a threaded portion 26 configured to threadedly engage a threaded portion 28 of the connector body 14. For example, the threaded portion 26 of the clamping ring 24 may be an interior threaded portion configured to mate with an exterior threaded portion of the legs 18 of the connector body 14. Alternatively, the securing element may be a screw having an exterior threaded portion configured to mate with an interior threaded portion of the legs 18 of the connector body 14.

The spinal stabilization system 10 may also include one or more, or a plurality of support constructs 30 positionable between and secured to vertebral connectors of the spinal stabilization system 10. As an illustrative example, the spinal stabilization system 10 shown in FIGS. 1 and 2 includes a support construct 30 extending between the vertebral connectors 12. It is noted, however, that one or more of the vertebral connectors 12 could be replaced with another style of vertebral connector, such as a pedicle screw or a lamina hook, if desired.

The support construct 30 may be constructed of a plurality of components in some instances. For instance, the support construct 30 may include a spacer 32, and a flexible member such as a flexible cord 34 extending through the spacer 32, as well as other components if desired.

In some embodiments, the spacer 32 may be an annular spacer having a lumen (not shown) extending from a first end to a second end of the spacer 32. For example, in some embodiments the spacer 32 may be a cylindrical member having a lumen extending therethrough. In other embodiments, the spacer 32 may be molded, extruded, or otherwise formed over and/or around the cord 34. In some embodiments, the spacer 32 may be formed from polycarbonate urethane (PCU), although it will be recognized that various other materials suitable for implantation within the human body and for providing stabilization of the spine while maintaining flexibility may be used. In other embodiments, the spacer 32 can be constructed of other materials such as metal, polymeric materials, or combinations of materials.

The cord 34 may extend from the opposing ends of the spacer 32. In one embodiment, the cord 34 may be formed from polyethylene-terephthalate (PET), although it will be recognized that various other materials suitable for implantation within the human body and for providing stabilization of the spine while maintaining flexibility may be used. In other embodiments, the cord 34 can be constructed of other flexible materials such as metal, polymeric materials, or combinations of flexible materials. It is noted that during a medical procedure any excess portions of the cord 34 extending from the spinal stabilization system 10 may be trimmed as desired to reduce and/or eliminate excess portions of the cord 34.

When implanted in a patient, the cord 34 of the spinal stabilization system 10 may limit the range of flexion of the spinal segment, whereas the spacer 32 may limit the range of extension of the spinal segment. For instance, the cord 34 may be placed in tension and the spacer 32 may be placed in compression when the support construct 30 is installed between the vertebral connectors 12.

The support construct 30 may also include inserts 36, 38 configured to be inserted into the channels 16 of the connector bodies 14 of the vertebral connectors 12 (or the pedicle screw 13, if used). An end of the cord 34 may be fixedly secured to an end insert 36 either indirectly or directly, such that the end of the cord 34 is locked from movement relative to the end insert 36. For example, the end of the cord 34 may be crimped, swaged, clamped, adhesively bonded, or pinned in a bore of the end insert 36, or otherwise attached to the end insert 36. In other embodiments, the end of the cord 34 may be indirectly attached to the end insert 36.

One possible embodiment of the end insert 36 is further illustrated in FIG. 2. The end insert 36, which may be considered a spool in some instances, may include a first flange 40 proximate a first end of the end insert 36, a second flange 42 proximate the second end of the end insert 36, and a medial portion 44 intermediate the first flange 40 and the second flange 42 and extending therebetween.

The free insert 38, which may be considered a spool in some instances, may be similar to the end insert 36 in many respects. One possible embodiment of the free insert 38 is also illustrated in FIG. 2. Similar to the end insert 36, the free insert 38 may include a first flange 40 proximate a first end of the free insert 38, a second flange 42 proximate the second end of the free insert 38, and a medial portion 44 intermediate the first flange 40 and the second flange 42 and extending therebetween. The free insert 38 may include a central lumen 46 extending longitudinally through the free insert 38 configured to slidably receive the cord 34 therethrough. Thus, the free insert 38 may be free to slide along a portion of the cord 34.

Although the support construct 30 is illustrated utilizing an end insert 36 and a free insert 38, in some instances, it may be desirable to substitute a free insert 38 for the end insert 36 and/or substitute an end insert 36 for the free insert 38.

As shown in FIG. 1, the inserts 36, 38 may be configured such that the medial portion 44 is positionable in the channel 16 of the connector body 14 of the vertebral connector 12 with the first flange 40 positioned exterior of the connector body 14 and facing the first side of the connector body 14 and the second flange 42 positioned exterior of the connector body 14 and facing the second side of the connector body 14. The inserts 36, 38 may be positioned in the channel 16 in a top-loaded fashion in which the inserts 36, 38 are moved into the channel 16 of the connector body 14 in a direction generally perpendicular to the longitudinal axis of the channel 16 of the connector body 14.

The inserts 36, 38 may be positioned on opposite sides of the spacer 32 and may have end surfaces configured to abut an end surface of the spacer 32. For instance, when assembled an end surface of the end insert 36 coupled with the first vertebral connector 12 may abut an end surface of the spacer 32 proximate the first end of the spacer 32 and an end surface of the free insert 38 coupled with the second vertebral connector 12 (or a pedicle screw or lamina hook, if used) may abut an end surface of the spacer 32 proximate the second end of the spacer 32. Thus, the spacer 32 may be positioned on the cord 34 between the end insert 36 and the free insert 38 with the cord 34 extending from the end insert 36, through the lumen of the spacer 32 and through the lumen 46 of the free insert 38.

The spinal stabilization system 10 may also include a locking collar 60 or other locking device configured to maintain a desired amount of tension in the cord 34 when assembled. For instance, the cord 34 may extend through a lumen 62 of the locking collar 60, which is placed in abutting contact with the free insert 38. With the components of the support construct 30 assembled on the cord 34, tension may be applied to the cord 34, and thus urge the inserts 36, 38 into compressive contact with the spacer 32. When the desired amount of tension has been applied to the cord 34, the set screw 64, or other locking feature of the locking collar 60, may be tightened against the cord 34 to lock the position of the cord 34 relative to the locking collar 60, and thus maintain the desired amount of tension in the cord 34. In other embodiments, the cord 34 may be tensioned, and then the cord 34 may be directly clamped in the channel of a vertebral connector (e.g., pedicle screw or lamina hook) with a set screw or other rotatable fastener engaged with the vertebral connector and pressing directly or indirectly against the cord 34, clamping the cord 34 to the vertebral connector. Tensioning of the cord 34, may be performed pre-operatively in instances in which the support construct 30 is pre-assembled, or the tensioning of the cord 34 may be performed intra-operatively, either in situ or prior to installation in the patient.

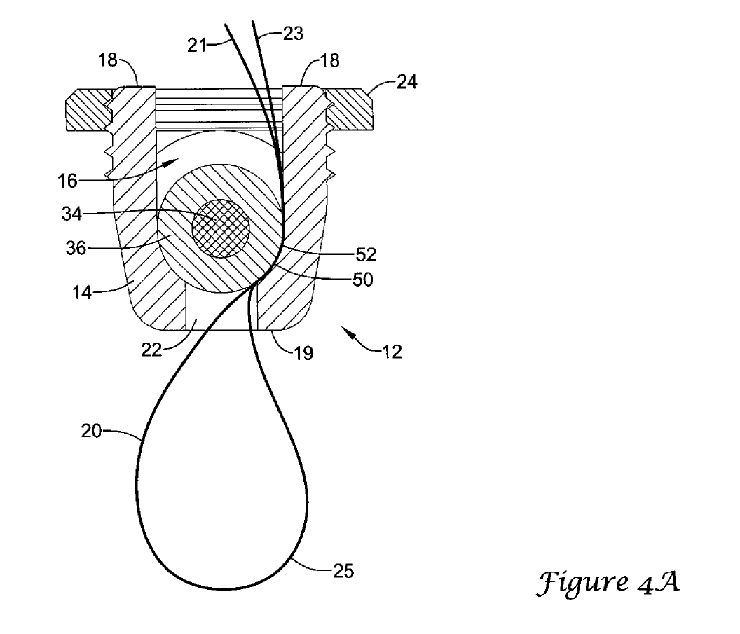

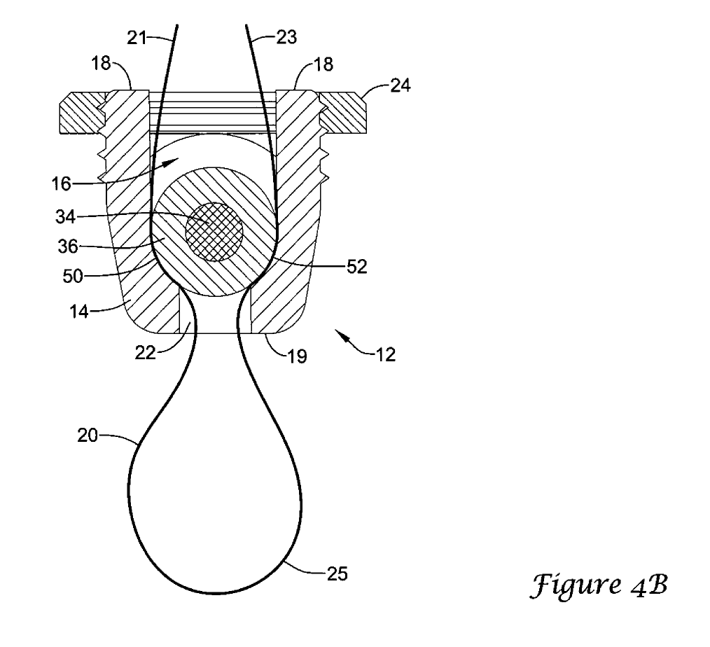

FIGS. 4A and 4B illustrate two alternate ways of routing the band 20 through the connector body 14 during installation of the vertebral connector 12 on a vertebra. In the first configuration, shown in FIG. 4A, both of the first and second portions 50, 52 of the band 20 are positioned through a passage between the medial portion 44 of the insert 36 and a surface of the channel 16 on a single side of the insert 36, with the loop portion 25 extending from the connector body 14. In such a configuration, the clamping ring 24 may be rotated relative to the connector body 14 in order to clamp the first and second portions 50, 52 of the band 20 between a surface of the insert 36 and the surface of the channel 16, thereby locking the band 20 from further movement. The vertebral connector 12 may be configured such that rotational engagement of the clamping ring 24 with the connector body 14 deflects the legs 18 of the connector body 14 toward one another (i.e., inward) and/or presses the clamping ring 24 against the insert 36, such as against the flanges 40, 42, to clamp the insert 36 in the channel 16 of the connector body 14 while simultaneously clamping the first and second portions 50, 52 of the band 20 to the connector body 14. Thus, during installation, the loop portion 25 of the band 20 may be passed around a bony portion of the vertebra, such as the transverse process, and then drawn against the bony portion a desired amount by tensioning the free ends 21, 23 of the band 20. With the desired amount of tension in the band 20 and the desired placement of the connector body 14, the clamping ring 24 may be rotatably engaged with the connector body 14 to simultaneously secure the insert 36 in the channel 16 of the connector body 14 and clamp the band 20 between the insert 36 and the connector body 14.

In the second configuration, shown in FIG. 4B, the first portion 50 of the band is positioned through a passage between the medial portion 44 of the insert 36 and a surface of the channel 16 on a first side of the insert 36 and the second portion 52 of the band 20 is positioned through a second passage between the medial portion 44 of the insert 36 and a surface of the channel 16 on an opposite side of the insert 36, with the loop portion 25 extending from the connector body 14. In such a configuration, the clamping ring 24 may be rotated relative to the connector body 14 in order to clamp the first and second portions 50, 52 of the band 20 between a surface of the insert 36 and the surface of the channel 16, thereby locking the band 20 from further movement. The vertebral connector 12 may be configured such that rotational engagement of the clamping ring 24 with the connector body 14 deflects the legs 18 of the connector body 14 toward one another (i.e., inward) and/or presses the clamping ring 24 against the insert 36, such as the flanges 40, 42, to clamp the insert 36 in the channel 16 of the connector body 14 while simultaneously clamping the first and second portions 50, 52 of the band 20 to the connector body 14. Thus, during installation, the loop portion 25 of the band 20 may be passed around a bony portion of the vertebra, such as the transverse process, and then drawn against the bony portion a desired amount by tensioning the free end portions 21, 23 of the band 20. With the desired amount of tension in the band 20 and the desired placement of the connector body 14, the clamping ring 24 may be rotatably engaged with the connector body 14 to simultaneously secure the insert 36 in the channel 16 of the connector body 14 and clamp the band 20 between the insert 36 and the connector body 14.

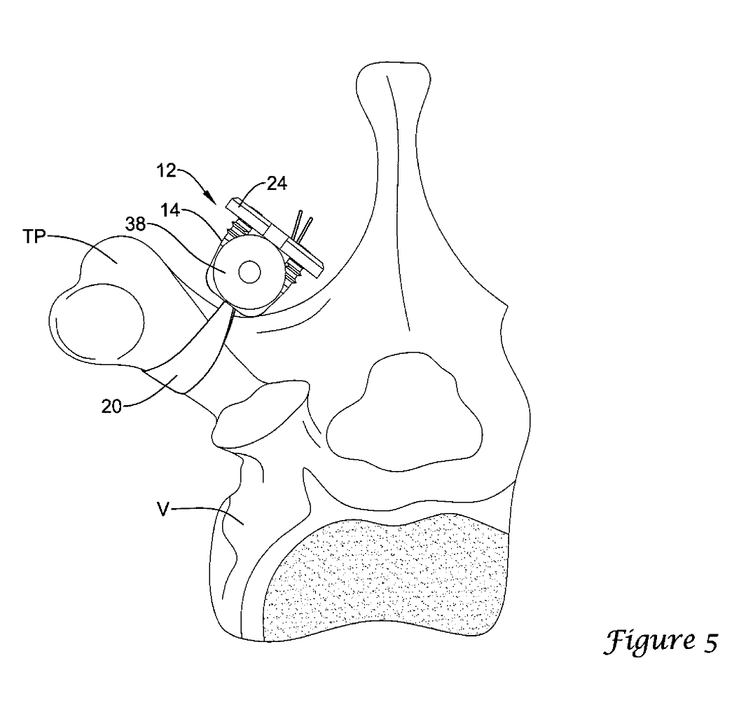

FIG. 5 illustrates the vertebral connector 12 secured to a transverse process TP of a vertebra V. As shown, the loop portion 25 of the band 20 may pass around the transverse process TP with the first and second end portions 21, 23 extending from the connector body 14 opposite the loop portion 25. As the first and second end portions 21, 23 are pulled, the loop portion 25 is cinched against the transverse process TP. While maintaining the connector body 14 in a desired position and while maintaining a desired amount of tension in the band 20, the vertebral connector 12 may be secured to the transverse process TP by rotatably engaging the clamping ring 24 with the connector body 14. Thus, the vertebral connector 12 may be secured to the transverse process TP of the vertebra V without invasively penetrating into the vertebra V and/or removing material from the vertebra V.

Accordingly, the vertebral connector 12 may be used to secure the support construct 30 of the spinal stabilization system 10 to a vertebra of a spinal column. It is noted that in other embodiments, the vertebral connector 12 may be replaced with another clamping device including a band for passing around a bony portion of the vertebra as disclosed in U.S. Pat. App. Pub. 2009/0105715, incorporated by reference herein, in order to secure the support construct 30 to a vertebra.

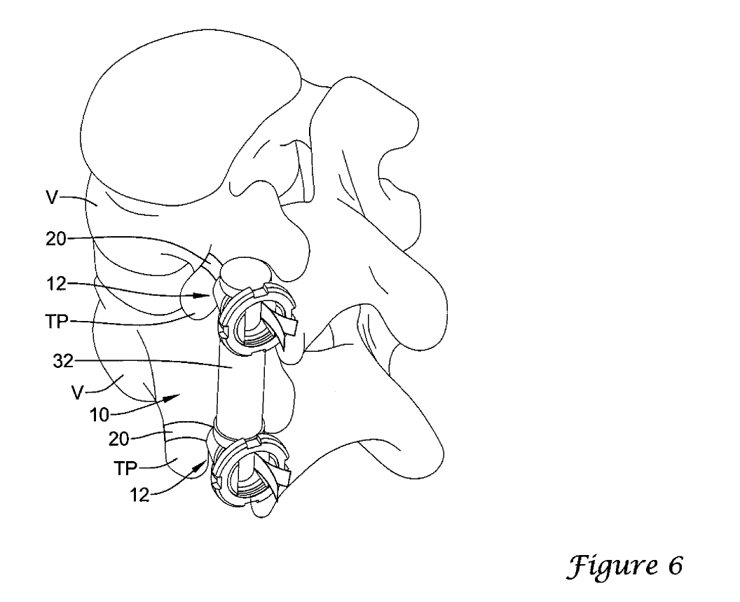

FIG. 6 is a perspective view of the spinal stabilization system 10 installed on a spinal segment of a spinal column. As shown in FIG. 6, the bands 20 of the first and second vertebral connectors 12 extend around the transverse processes TP of first and second vertebrae, respectively, to secure the spinal stabilization system 10 to the segment of the spinal column. The support construct 30 may be secured to the vertebral connectors 12 such that the spinal stabilization system 10 is oriented in a generally superior-inferior orientation on the posterior portion of the spinal column.

In some instances, the spinal stabilization system 10 may be installed unilaterally (i.e., on a single side of the sagital plane of the spine), while in other instances multiple spinal stabilization systems 10 may be installed multi-laterally (i.e., on multiple sides of the sagital plane of the spine). For instance, a first spinal stabilization system 10 may be installed along one side of the spinal column, while a second or additional spinal stabilization system 10 may be installed along a second side of the spinal column.

The spinal stabilization system 10 may be installed on the spinal segment in a number of ways. For example, the band 20 of the first vertebral connector 12 may be routed around a transverse process TP (or other bony anatomy) of a first vertebra and the free ends may be passed through the opening 22 of the connector body 14 with the loop portion 25 encircling the transverse process TP. The end insert 36 may then be loaded into the channel 16 of the connector body 14. Upon tensioning the band 20 a sufficient amount to tighten the band 20 around the transverse process TP, the clamping ring 24 may be rotatably engaged with the connector body 14 to press the end insert 36 against the connector body 14, and thus clamp the end insert 36 to the vertebral connector 12. Simultaneously, the band 20 may be clamped to the connector body 14, as described above, to secure the vertebral connector 12 to the transverse process TP.

The second vertebral connector 12 may be similarly secured to a transverse process TP (or other bony anatomy) of a second vertebra, by routing the band 20 of the second vertebral connector 12 around the transverse process TP of the second vertebra with the free ends of the band 20 passing through the opening 22 of the connector body 14, with the loop portion 25 encircling the transverse process TP. The free insert 38 may then be loaded into the channel 16 of the connector body 14, either with the cord 34 previously positioned through the lumen 46 of the free insert 38 or subsequently positioned therethrough. Upon tensioning the band 20 a sufficient amount to tighten the band 20 around the transverse process TP, the fastener 24 may be rotatably engaged with the connector body 14 to press the free insert 38 against the connector body 14, and thus clamp the free insert 38 to the vertebral connector 12. Simultaneously, the band 20 may be clamped to the connector body 14, as described above, to secure the vertebral connector 12 to the transverse process TP.

The spacer 32 may be positioned between the end insert 36 and the free insert 38, with the cord 34 extending from the end insert 36, through the spacer 32, and through the lumen 46 of the free insert 38. The spacer 32 may be positioned between the inserts 36, 38 pre-operatively in instances in which the support construct 30 is pre-assembled, or the spacer 32 may be positioned between the inserts 36, 38 intra-operatively, either in situ or prior to installation in the patient.

In instances in which the support construct 30 is installed in situ, tension may then be applied to the cord 34 to compress the inserts 36, 38 against the spacer 32. Once the cord 34 has been appropriately tensioned, the locking collar 60, or other locking means, may be secured to the cord 34 to maintain the desired amount of tension in the cord 34. In instances in which the support construct 30 is pre-assembled, either pre-operatively or intra-operatively, the cord 34 may be similarly tensioned and subsequently locked with the locking collar 60, or other locking means. The cord 34 may then be trimmed to remove any excess length of the cord 34.

FIG. 7 illustrates an exemplary multi-level spinal stabilization system 110 for stabilizing a portion of a spinal column utilizing similar components used with the spinal stabilization system 10. Such a multi-level system may be secured to three or more vertebrae of a spinal column. The spinal stabilization system 110, in some instances, may provide dynamic stabilization to a spinal segment, preserving and/or allowing for a range of motion of the spinal segment. For example, in some instances the spinal stabilization system 110 may allow for a range of motion of greater than 0 degrees to about 15 degrees or more, greater than 0 degrees to about 10 degrees or more, or greater than 0 degrees to about 5 degrees or more between adjacent vertebrae. In some instances, the spinal stabilization system 110 may allow for 2 degrees or more, 5 degrees or more, 10 degrees or more, or 12 degrees or more of motion between adjacent vertebrae.

In some instances, the spinal stabilization system 110 may be installed unilaterally (i.e., on a single side of the sagital plane of the spine), while in other instances multiple spinal stabilization systems 110 may be installed multi-laterally (i.e., on multiple sides of the sagital plane of the spine). For instance, a first spinal stabilization system 110 may be installed along one side of the spinal column, while a second or additional spinal stabilization system 110 may be installed along a second side of the spinal column.

As shown in FIG. 7, the spinal stabilization system 110 may include three or more vertebral connectors for securing the spinal stabilization system 110 to vertebrae along the spinal column. One or more of the vertebral connectors may be vertebral connectors 12 which include a band 20, or other flexible member, for wrapping around a bony structure of a vertebra (e.g. transverse process, costal process, spinous process, vertebral arch, etc.) to secure the vertebral connector 12 to a vertebra of the spinal column. Thus, the vertebral connector 12 may be secured to a vertebra without invasively removing material from the vertebra and/or penetrating into the vertebra.

However, one or more of the vertebral connectors may be pedicle screws 13 (one of which is shown in FIG. 7), vertebral hooks (e.g., laminar hooks) or other types of fastening members for attachment to a bony structure such as a vertebra of the spinal column. For instance, the pedicle screw 13 may be screwed into the pedicle of a vertebra to secure the stabilization system 110 to the spinal column while one or more of the other vertebral connectors of the stabilization system 110 may be a vertebral connector 12 including a band 20 for securing the vertebral connector 12 to the vertebra. It is noted however that the pedicle screw 13 may be replaced with another vertebral connector 12 having a band 20 for securing the vertebral connector 12 to a vertebra, if desired. Although the pedicle screw 13 is shown positioned intermediate the vertebral connectors 12, the pedicle screw 13, if used with the stabilization system 110, may be located at any position along the stabilization system 110.

In the case of a pedicle screw 13 used to secure the spinal stabilization system 110 to a vertebra, the pedicle screw 13 may include a connector body 14′ and a threaded shaft 15, extending from the connector body 14′. The connector body 14′ may include first and second legs 18′ extending from a base portion 19′ defining a channel 16′ therebetween extending through the connector body 14′ from a first side of the connector body 14′ to a second side of the connector body 14′ opposite the first side. The shaft 15 may be configured to be installed into a bony region of a vertebra of the spinal column. For example, the shaft 15 may be installed into a pedicle of a vertebra, or other region of a vertebra.

The pedicle screw 13 may include a securing element, such as a threaded fastener 24′ (e.g., a set screw, cap) configured to rotatably engage the connector body 14′ to secure a portion of a support construct 30 to the pedicle screw 13. For example, the threaded fastener 24′ may include threads which mate with threads formed in the connector body 14′. In other embodiments, the fastener 24′ may include one or more flanges, cam surfaces, or other engagement features that engage with one or more channels, grooves, surfaces, or other engagement features of the connector body 14′ through rotation of the fastener 24′. The fastener 24′ may be rotatably engaged between spaced apart legs 18′ of the connector body 14′ which define the channel 16′ therebetween.

The spinal stabilization system 110 may also include one or more, or a plurality of support constructs 30 positionable between and secured to vertebral connectors of the spinal stabilization system 110. As an illustrative example, the spinal stabilization system 110 shown in FIG. 7 includes a first support construct 30 extending between the first vertebral connector 12 and the pedicle screw 13 and a second support construct 30 extending between the pedicle screw 13 and the second vertebral connector 12. It is noted, however, that one or more of the vertebral connectors 12 could be replaced with another style of vertebral connector, such as a pedicle screw or a lamina hook, and/or the pedicle screw 13 could be replaced with a vertebral connector 12 including a band 20 for securing the vertebral connector 12 to a vertebra, if desired.

The support constructs 30 may be constructed of a plurality of components in some instances. For instance, the support constructs 30 may include a spacer 32, and a flexible member such as a flexible cord 34 extending through each spacer 32. The support constructs 30 may also include inserts 36, 38 configured to be inserted into the channels 16, 16′ of the connector bodies 14, 14′ of the vertebral connectors 12 (or the pedicle screw 13, if used). An end of the cord 34 may be fixedly secured to an end insert 36 either indirectly or directly, such that the end of the cord 34 is locked from movement relative to the end insert 36. The free inserts 38 may include a central lumen 46 extending longitudinally through the free inserts 38 configured to slidably receive the cord 34 therethrough. Thus, the free inserts 38 may be free to slide along a portion of the cord 34. It is noted that in some instances it may be desirable to substitute a free insert 38 for the end insert 36 and/or substitute an end insert 36 for one of the free inserts 38.

The spinal stabilization system 110 may also include a locking collar 60 or other locking device configured to maintain a desired amount of tension in the cord 34 when assembled. For instance, the cord 34 may extend through a lumen 62 of the locking collar 60, which is placed in abutting contact with the free insert 38. With the components of the support constructs 30 assembled on the cord 34, tension may be applied to the cord 34, and thus urge the inserts 36, 38 into compressive contact with the spacers 32. When the desired amount of tension has been applied to the cord 34, the set screw 64, or other locking feature of the locking collar 60, may be tightened against the cord 34 to lock the position of the cord 34 relative to the locking collar 60, and thus maintain the desired amount of tension in the cord 34. In other embodiments, the cord 34 may be tensioned, and then the cord 34 may be directly clamped in the channel of a vertebral connector (e.g., pedicle screw or lamina hook) with a set screw or other rotatable fastener engaged with the vertebral connector and pressing directly or indirectly against the cord 34, clamping the cord 34 to the vertebral connector. Tensioning of the cord 34, may be performed pre-operatively in instances in which the support constructs 30 are pre-assembled, or the tensioning of the cord 34 may be performed intra-operatively, either in situ or prior to installation in the patient.

The spinal stabilization system 110 may be secured to the spinal column by cinching the bands 20 of the vertebral connectors 12 around the transverse processes TP of the respective vertebrae to secure the spinal stabilization system 110 to the segment of the spinal column. If a pedicle screw 13 is used, the pedicle screw 13 may be screwed into a bony portion of a chosen vertebra. The support constructs 30 may be secured to the vertebral connectors 12 (and pedicle screw 13 if present) such that the spinal stabilization system 110 is oriented in a generally superior-inferior orientation on the posterior portion of the spinal column.

The inserts 36, 38 may be loaded into the channels 16, 16′ of the connector body 14, 14′, either with the cord 34 previously positioned through the lumens 46 of the free inserts 38 or subsequently positioned therethrough, and clamped thereto with a rotatable fastener 24, 24′. Simultaneously, the bands 20 of the vertebral connectors 12 may be clamped to the connector bodies 14, as described above, to secure the vertebral connectors 12 to the transverse process TP of the respective vertebra.

The spacers 32 may be positioned between adjacent inserts 36, 38 with the cord 34 extending from the end insert 36, through the first spacer 32, through the lumen 46 of the first free insert 38, through the second spacer 32, and through the lumen 46 of the second free insert 38. The cord 34 may be tensioned a desired amount to exert a compressive force on each spacer 32 by the inserts 36, 38. Once the cord 34 has been appropriately tensioned, the locking collar 60, or other locking means, may be secured to the cord 34 to maintain the desired amount of tension in the cord 34. The cord 34 may be trimmed to remove any excess length of the cord 34.

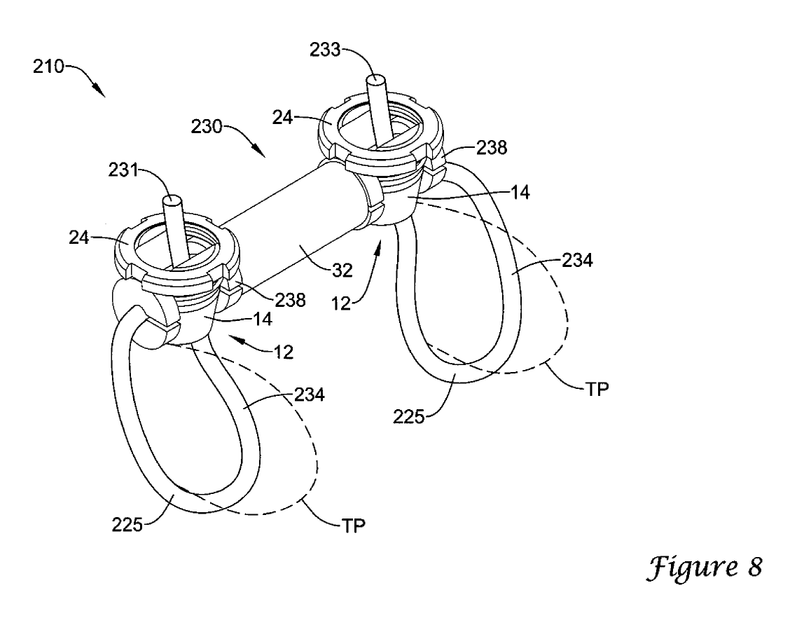

FIG. 8 illustrates another spinal stabilization system 210 for stabilizing a portion of a spinal column utilizing similar components used with the spinal stabilization system 10. Such a system may be secured to adjacent vertebrae of a spinal column to provide stabilization to the spinal segment. The spinal stabilization system 210, in some instances, may provide dynamic stabilization to a spinal segment, preserving and/or allowing for a range of motion of the spinal segment. For example, in some instances the spinal stabilization system 210 may allow for a range of motion of greater than 0 degrees to about 15 degrees or more, greater than 0 degrees to about 10 degrees or more, or greater than 0 degrees to about 5 degrees or more between adjacent vertebrae. In some instances, the spinal stabilization system 210 may allow for 2 degrees or more, 5 degrees or more, 10 degrees or more, or 12 degrees or more of motion between adjacent vertebrae.

In some instances, the spinal stabilization system 210 may be installed unilaterally (i.e., on a single side of the sagital plane of the spine), while in other instances multiple spinal stabilization systems 210 may be installed multi-laterally (i.e., on multiple sides of the sagital plane of the spine). For instance, a first spinal stabilization system 210 may be installed along one side of the spinal column, while a second or additional spinal stabilization system 210 may be installed along a second side of the spinal column.

As shown in FIG. 8, the spinal stabilization system 210 may include one or more vertebral connectors for securing the spinal stabilization system 210 to one or more vertebrae of the spinal segment to be stabilized. One or more of the vertebral connectors may be vertebral connectors 12 configured to be secured to a bony structure of a vertebra (e.g. transverse process, costal process, spinous process, vertebral arch, etc.) to secure the vertebral connector 12 to a vertebra of the spinal column without invasively removing material from the vertebra and/or penetrating into the vertebra. The spinal stabilization system 210 is shown in FIG. 8, secured to the transverse process TP (shown in dashed lines) of first and second adjacent vertebrae. The vertebral connector 12 may include a connector body 14 and a clamping ring 24 rotatably engaged with the connector body 14, as discussed above.

However, one or more of the vertebral connectors may be pedicle screws 13 (one of which is shown in FIG. 7), vertebral hooks (e.g., laminar hooks) or other types of fastening members for attachment to a bony structure such as a vertebra of the spinal column, if desired. For instance, the pedicle screw 13 may be screwed into the pedicle of a vertebra to secure the stabilization system 210 to the spinal column while one or more other vertebral connectors of the stabilization system 210 may be a vertebral connector 12.

The spinal stabilization system 210 may also include one or more, or a plurality of support constructs 30 positionable between and secured to vertebral connectors of the spinal stabilization system 210. As an illustrative example, the spinal stabilization system 210 shown in FIG. 8 includes a support construct 30 extending between the vertebral connectors 12. It is noted, however, that one or more of the vertebral connectors 12 could be replaced with another style of vertebral connector, such as a pedicle screw or a lamina hook, if desired.

The support construct 30 may include a spacer 32, as described above, having a lumen extending therethrough sized to receive a flexible member 234, such as a flexible cord or band. The flexible member 234, further described herein, may also be used to pass around a bony structure of a vertebra (e.g. transverse process, costal process, spinous process, vertebral arch, etc.) to secure one or more of the vertebral connectors 12 to a vertebra of the spinal column without invasively removing material from the vertebra and/or penetrating into the vertebra, instead of a separate band 20 described above with respect to the spinal stabilization system 10 illustrated in FIG. 1.

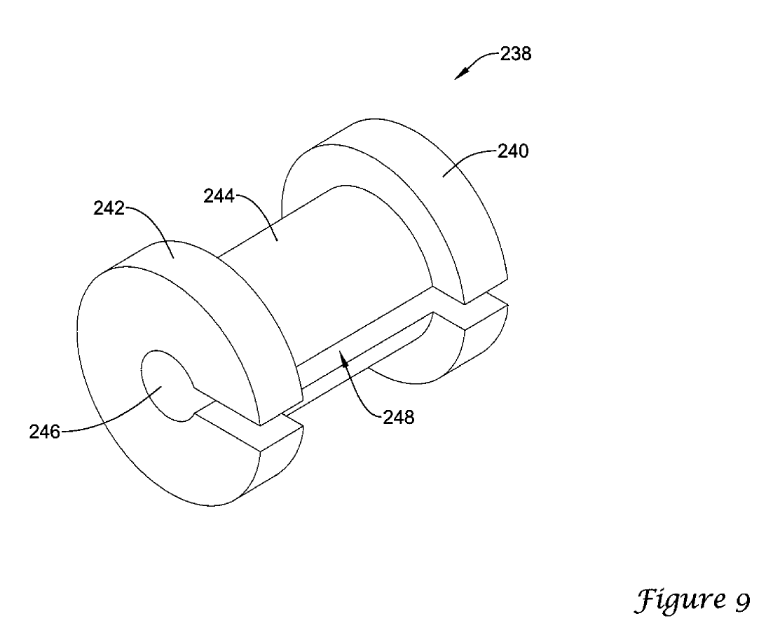

The support construct 30 may also include inserts 238 configured to be inserted into the channels 16 of the connector bodies 14 of the vertebral connectors 12 (or the pedicle screw 13, if used). The insert 238, shown in FIG. 9, may be configured to be selectively secured or clamped to the flexible member 234.

It is noted that in some instances it may be desirable to substitute a free insert 38 and/or an end insert 36, as described above, for one or more of the inserts 238 of the spinal stabilization system 210. Additionally, in some instances, one or more inserts 238 may be substituted for one or more of the end inserts 36 and free inserts 38, of the embodiments described above.

Similar to the end insert 36 and the free insert 38, the insert 238 may include a first flange 240 proximate a first end of the insert 238, a second flange 242 proximate the second end of the insert 238, and a medial portion 244 intermediate the first flange 240 and the second flange 242 and extending therebetween. The insert 238 may include a central lumen 246 extending longitudinally through the insert 238 configured to receive the flexible member 234 therethrough.

Although the support construct 230 is illustrated utilizing inserts 238 which may selectively secure the flexible member 234 from longitudinal movement through the inserts 238, in some instances, it may be desirable to substitute a free insert 38 and/or an end insert 36, if desired.

Similar to the inserts 36, 38 described above, the inserts 238 may be configured such that the medial portion 244 is positionable in the channel 16 of the connector body 14 of the vertebral connector 12 with the first flange 240 positioned exterior of the connector body 14 and facing the first side of the connector body 14 and the second flange 242 positioned exterior of the connector body 14 and facing the second side of the connector body 14. The inserts 238 may be positioned in the channel 16 in a top-loaded fashion in which the inserts 238 are moved into the channel 16 of the connector body 14 in a direction generally perpendicular to the longitudinal axis of the channel 16 of the connector body 14.

The inserts 238 may be positioned on opposite sides of the spacer 32 and may have end surfaces configured to abut an end surface of the spacer 32. For instance, when assembled an end surface of the first insert 238 coupled with the first vertebral connector 12 may abut an end surface of the spacer 32 proximate the first end of the spacer 32 and an end surface of the second insert 238 coupled with the second vertebral connector 12 may abut an end surface of the spacer 32 proximate the second end of the spacer 32. Thus, the spacer 32 may be positioned on the flexible member 234 between the inserts 238 with the flexible member 234 extending through the lumen of the spacer 32 and through the lumens 246 of the inserts 238.

The insert 238 may be configured to selectively secure the flexible member 234, extending through the lumen 246, from longitudinal movement relative to the insert 238. For instance, the insert 238 may be configured to selectively clamp the flexible member 234 in the lumen 246. For example, in some instances the insert 238 may be compressible such that a force applied to the insert 238 presses the insert 238 against the flexible member 234, generating sufficient frictional forces between the flexible member 234 and the insert 238 to inhibit longitudinal movement of the flexible member 234 through the lumen 246 of the insert 238. In some instances the insert 238 may be formed of a malleable material, such as a polymeric or metallic material, which may be plastically deformed or elastically deformed into frictional contact with the flexible member 234.

In the exemplary embodiment of the insert 238 shown in FIG. 9, the insert 238 may include a longitudinal slot 248 extending along the length of the insert 238 from the first end to the second end of the insert 238. In some instances the slot 248 may extend the entire length of the insert 238, while in other instances the slot 248 may extend along only a portion of the length of the insert 238. The slot 248 may extend radially inward from the exterior of the insert 238 to the central lumen 246. The slot 248 may allow portions of the insert 238 to be compressed toward one another, thereby reducing the cross-sectional dimensions (e.g., diameter) of the lumen 246 of the insert 238 in order to clamp the flexible member 234 in the lumen 246.

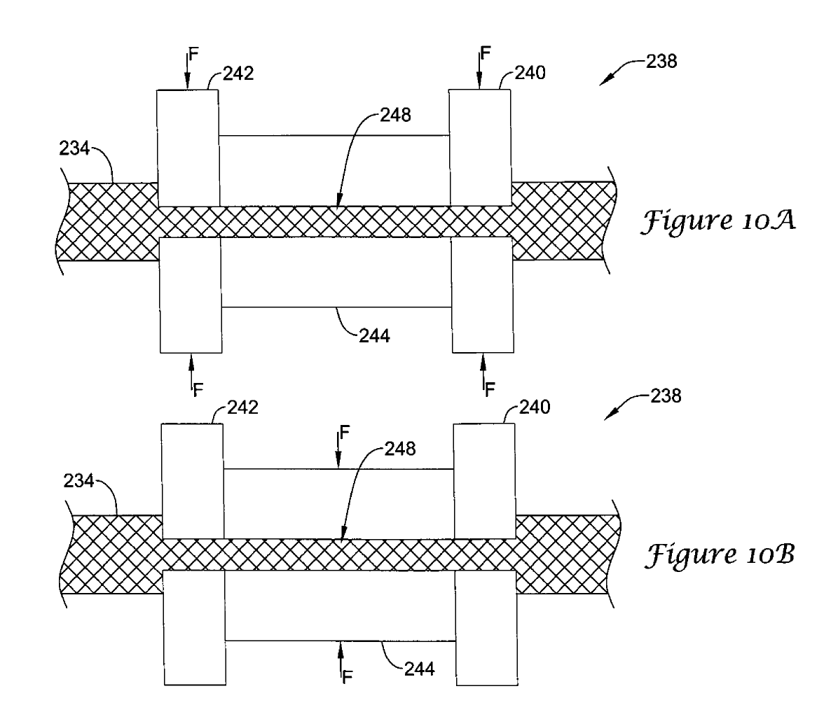

For instance, as shown in FIG. 10A, a radially inward force F may be exerted onto the flanges 240, 242 of the insert 238 to clamp the flexible member 234 in the lumen 246 and/or a radially inward force may be exerted onto the medial portion 244 of the insert 238 to clamp the flexible member 234 in the lumen 246, as shown in FIG. 10B.

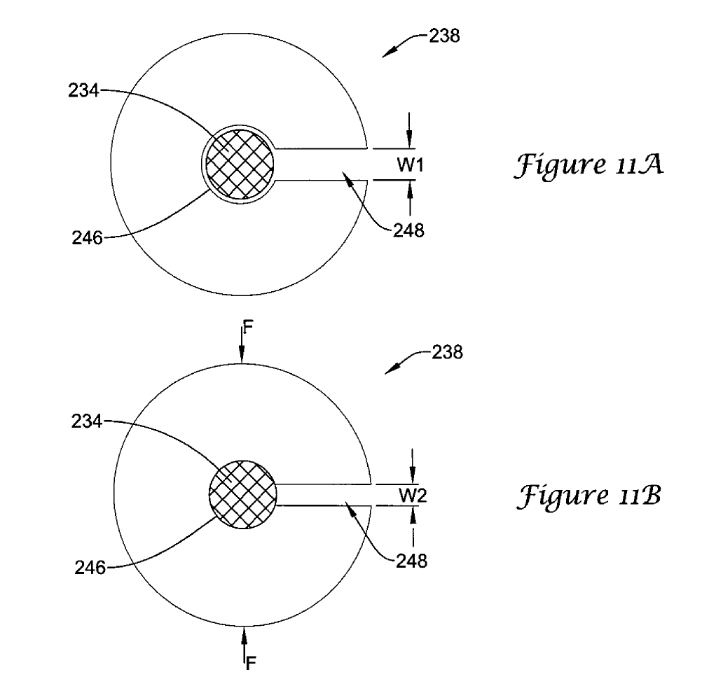

As shown in FIG. 11A, in an equilibrium condition in which the insert 238 is not subjected to external forces, the lumen 246 may be sized to allow free longitudinal movement of the flexible member 234 therethrough. When the clamping force F is applied to the insert 238, the width of the slot 248 may be reduced from a first width W1, shown in FIG. 11A, to a second width W2, shown in FIG. 11B. Reducing the width of the slot 248 correspondingly reduces the size (e.g., diameter) of the lumen 246 sufficiently such that the inner surface of the insert 238 defining the lumen 246 compresses against the flexible member 234, clamping the flexible member 234 in the lumen 246. In some instances, the inner surface of the insert 238 may include any mechanical gripping means such as, but not limited to, one or more threads, ribs, projecting grooves, teeth, posts, spikes, surface roughenings, knurlings, and/or serrations or combinations thereof. The mechanical gripping means may increase the purchase of the insert 238 with the flexible member 234.

In other embodiments, the flexible member 234 may be tensioned, and then the flexible member 234 may be directly clamped in the channel of a vertebral connector with a set screw or other rotatable fastener engaged with the vertebral connector and pressing directly or indirectly against the flexible member 234, clamping the flexible member 234 to the vertebral connector.

The spinal stabilization system 210 may be secured to the spinal column by cinching the flexible member 234 around the transverse processes TP of the respective vertebrae to secure the spinal stabilization system 210 to the segment of the spinal column. If a pedicle screw 13 is used, the pedicle screw 13 may be screwed into a bony portion of a chosen vertebra. The support construct 230 may be secured to the vertebral connectors 12 (and pedicle screw 13 if present) such that the spinal stabilization system 210 is oriented in a generally superior-inferior orientation on the posterior portion of the spinal column.

The inserts 238 may be loaded into the channels 16 of the connector bodies 14 either with the flexible member 234 previously positioned through the lumens 246 of the inserts 238 or subsequently positioned therethrough.

The spacer 32 may be positioned between adjacent inserts 238 with the flexible member 234 extending through the lumen 246 of the first insert 238, through the spacer 32, and through the lumen 246 of the second insert 238. A first end portion 231 of the flexible member 234 may be routed around a transverse process TP (or other bony anatomy) of a first vertebra and passed through the opening 22 of the connector body 14 of the first vertebral connector 12 with a loop portion of the flexible member 234 encircling the transverse process TP of the first vertebra. Similarly, a second end portion 233 of the flexible member 234 may be routed around a transverse process TP (or other bony anatomy) of a second vertebra and passed through the opening 22 of the connector body 14 of the second vertebral connector 12 with a loop portion of the flexible member 234 encircling the transverse process TP of the second vertebra.

The flexible member 234 may be tensioned a desired amount to exert a compressive force on the spacer 32 by the inserts 238. The flexible member 234 may be cinched around the transverse processes TP of the vertebrae to secure the spinal stabilization system 210 to the segment of the spinal column. Once the flexible member 234 has been appropriately tensioned and cinched around the transverse processes, the flexible member 234 may be clamped thereto with rotatable fasteners 24 engaged with the connector bodies 14. Rotation of the fasteners 24 may simultaneously apply a clamping force to the inserts 238 to compress the inserts 238 thereby clamping the flexible member 234 in the lumens 246 of the inserts 238, and clamp the end portions 231, 233 of the flexible member 234 between a surface of a respective insert 238 and a surface of the channel 16 of a respective connector body 14, thereby locking the flexible member 234 from further movement. The flexible member 234 may be trimmed to remove any excess length of the flexible member 234.

Thus, the flexible member 234 may extend continuously through the components of the support construct 230 (e.g., inserts 238 and spacer 32), as well as extend around the transverse processes TP of the vertebrae to secure the spinal stabilization system 210 to the vertebrae.

Accordingly, it can be appreciated that the afore described spinal stabilization systems may be secured to one or more vertebrae of a spinal segment of a spinal column without the use of a pedicle screw in instances in which the use of a pedicle screw is not desired and/or not possible.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A spinal stabilization system for a spinal column, the system comprising:
a first vertebral connector configured to be secured to a first vertebra;
a second vertebral connector configured to be secured to a second vertebra; and
a support construct positionable between the first vertebral connector and the second vertebral connector for providing stabilization between the first and second vertebrae, the support construct including a flexible cord extending from an insert and a spacer including a lumen through which the cord extends through;
wherein the first vertebral connector includes a connector body having a channel for receiving the insert therein and an elongate band having a first end, a second end and a portion configured to pass around a bony portion of the first vertebra to secure the first vertebral connector to the first vertebra.

2. The system of claim 1, wherein the insert is compressed against the flexible cord to create frictional forces between the insert and the flexible cord to impede relative movement between the flexible cord and the insert.

3. The system of claim 2, wherein the insert includes a lumen therethrough for receiving the flexible cord such that a clamping force exerted against the insert clamps the flexible cord in the lumen of the insert.

4. The system of claim 3, wherein the insert includes a longitudinal slot extending along the insert from an exterior of the insert to the lumen of the insert.

5. The system of claim 1, wherein the elongate band passes through the channel of the connector body such that the elongate band can be clamped between the insert and the connector body.

6. The system of claim 5, wherein the first vertebral connector includes a threaded fastener threadedly engaging the connector body to simultaneously secure the insert in the channel of the connector body and clamp the elongate band between the insert and the connector body.

7. The system of claim 1, wherein the insert includes a first flange, a second flange and a medial portion extending between the first flange and the second flange, the medial portion of the insert configured to be positioned in the channel of the connector body with the first flange positioned on a first side of the connector body and the second flange positioned on a second side of the connector body opposite the first side.

8. The system of claim 1, wherein the flexible cord is fixedly secured to the insert.

9. The system of claim 1, wherein the second vertebral connector includes a connector body having a channel and an elongate band having a first end, a second end and a portion configured to pass around a bony portion of the second vertebra to secure the second vertebral connector to the second vertebra.

10. The system of claim 9, wherein the support construct includes a second insert disposed on the flexible cord such that the spacer is positioned between the first and second inserts, the second insert being configured to be received in the channel of the connector body of the second vertebral connector.

11. The system of claim 10, wherein the second vertebral connector includes a threaded fastener threadedly engaging the connector body of the second vertebral connector to simultaneously secure the second insert in the channel of the connector body of the second vertebral connector and clamp the elongate band of the second vertebral connector between the second insert and the connector body of the second vertebral connector.

12. A spinal stabilization system for a spinal column, the system comprising:
a first vertebral connector configured to be secured to a first vertebra, the first vertebral connector including a connector body having a channel extending through the connector body from a first end of the connector body to a second end of the connector body and a flexible component configured to pass around a bony portion of the first vertebra to secure the first vertebral connector to the first vertebra;
a second vertebral connector configured to be secured to a second vertebra, the second vertebral connector including a connector body having a channel extending through the connector body from a first end of the connector body to a second end of the connector body;
a first insert including at least a portion positionable in the channel of the connector body of the first vertebral connector;
a second insert including at least a portion positionable in the channel of the connector body of the second vertebral connector;
a spacer positionable between the first insert and the second insert; and
a flexible member extending through the spacer from the first insert to the second insert.

13. The system of claim 12, wherein the flexible component of the first vertebral connector passes through the channel of the connector body of the first vertebral connector such that the flexible component can be clamped between the first insert and the connector body of the first vertebral connector.

14. The system of claim 13, wherein the first vertebral connector includes a threaded fastener threadedly engaging the connector body of the first vertebral connector to simultaneously secure the first insert in the channel of the connector body of the first vertebral connector and clamp the flexible component between the first insert and the connector body of the first vertebral connector.

15. The system of claim 12, wherein the flexible member is fixedly attached to the first insert and the flexible member is slidably disposed through a lumen of the second insert.

16. The system of claim 12, wherein the flexible component of the first vertebral connector is a continuous portion of the flexible member.

17. The system of claim 12, wherein the second vertebral connector includes a flexible component configured to pass around a bony portion of the second vertebra to secure the second vertebral connector to the second vertebra.

18. The system of claim 17, wherein the flexible component of the first vertebral connector and the flexible component of the second vertebral connector are both continuous portions of the flexible member.

19. A method of stabilizing a spinal segment of a spinal column, the method comprising:
securing a first vertebral connector to a first vertebra of the spinal column by passing a flexible band of the first vertebral connector around a bony portion of the first vertebra and securing the flexible band to a connector body of the first vertebral connector;
securing a second vertebral connector to a second vertebra of the spinal column; and
coupling a stabilization construct between the first vertebral connector and the second vertebral connector, the stabilization construct configured to allow a range of flexion and extension of the spinal segment.

20. The method of claim 19, wherein the stabilization construct includes a cord for limiting the range of flexion of the spinal segment and a spacer for limiting the range of extension of the spinal segment

21. The method of claim 20, wherein the cord is placed in tension between the first and second vertebral connectors and the spacer is placed in compression between the first and second vertebral connectors.

22. The method of claim 19, wherein the flexible band is passed around a transverse process of the first vertebra to secure the first vertebral connector to the first vertebra.

23. The method of claim 22, wherein the second vertebral connector is secured to the second vertebra by passing a flexible band of the second vertebral connector around a bony portion of the second vertebra and securing the flexible band to a connector body of the second vertebral connector.

24. The method of claim 19, wherein the elongate band is clamped between a component of the stabilization construct and the connector body of the first vertebral connector simultaneously as the stabilization construct is coupled to the first vertebral connector with a threaded fastener.
